SEQUENCE LISTING (25 sequences)

SEQ ID NO: 1 - DNA, 20 bp, Artificial Sequence
  Primer
cctgctctgccgcttcacgc

SEQ ID NO: 2 - DNA, 21 bp, Artificial Sequence
  Primer
gggccgtggcagtcgcatccc

SEQ ID NO: 3 - DNA, 28 bp, Artificial Sequence
  Synthetic construct
tuuuuuctatccttatttttaaggatag

SEQ ID NO: 4 - DNA, 24 bp, Artificial Sequence
  Synthetic construct
tuuuuuagctaggttttcctagct

SEQ ID NO: 5 - DNA, 24 bp, Artificial Sequence
  Synthetic construct
tuuuuugcagcgatttttcgctgc

SEQ ID NO: 6 - DNA, 30 bp, Artificial Sequence
  Synthetic Construct
tuuuuugagactcgrcttttgacgagtctc

SEQ ID NO: 7 - DNA, 34 bp, Artificial Sequence
  Synthetic construct
tuuuuuctatccttaacgttttcgttaaggatag

SEQ ID NO: 8 - DNA, 30 bp, Artificial Sequence
  Synthetic construct
tuuuuuacacttccggttttccggaagtct

SEQ ID NO: 9 - DNA, 31 bp, Artificial Sequence
  Synthetic construct
tuuuuuctatccttaacgncgttaaggatag

SEQ ID NO: 10 - DNA, 34 bp, Artificial Sequence
  Synthetic construct
tuuuuuctatccttaacgnnnncgttaaggatag

SEQ ID NO: 11 - DNA, 36 bp, Artificial Sequence
  Synthetic construct
tuuuuuctatccttaacgttttttcgttaaggatag

SEQ ID NO: 12 - DNA, 40 bp, Artificial Sequence
  Synthetic construct
tuuuuuctatccttaacgttttttttttcgttaaggatag

SEQ ID NO: 13 - DNA, 34 bp, Artificial Sequence
  Synthetic construct
tuuuuuctatccttaacnttttcgttaaggatag

SEQ ID NO: 14 - DNA, 34 bp, Artificial Sequence; identical to SEQ ID NO: 13
  Synthetic construct

SEQ ID NO: 15 - DNA, 34 bp, Artificial Sequence
  Synthetic construct
tuuuuuatctccttaacnttttcgttaaggagat

SEQ ID NO: 16 - DNA, 34 bp, Artificial Sequence
  Synthetic construct
tuuuuuctntccttnncgttttcgttaaggatag

SEQ ID NO: 17 - DNA, 34 bp, Artificial Sequence
  Synthetic construct
tauggactatccttaacgttttcgttaaggatag

SEQ ID NO: 18 - DNA, 34 bp, Artificial Sequence
  Synthetic construct
tuuugactatccttaacgttttcgttaaggatag

SEQ ID NO: 19 - DNA, 34 bp, Artificial Sequence
  Synthetic construct
ttntttctatccttaacgttttcgttaaggatag

SEQ ID NO: 20 - DNA, 34 bp, Artificial Sequence; identical to SEQ ID NO: 19
  Synthetic construct

SEQ ID NO: 21 - DNA, 34 bp, Artificial Sequence
  Synthetic construct
ttntttatctccttaacgttttcgttaaggagat

SEQ ID NO: 22 - DNA, 34 bp, Artificial Sequence
  Synthetic construct
tnnnttctatccttaacgttttcgttaaggatag

SEQ ID NO: 23 - DNA, 34 bp, Artificial Sequence; identical to SEQ ID NO: 22
  Synthetic construct

SEQ ID NO: 24 - DNA, 34 bp, Artificial Sequence
  Synthetic construct
tnnnttatctccttaacgttttcgttaaggagat

SEQ ID NO: 25 - protein, 587 aa, Bacillus stearothermophilus
AEGEKPLEEMEFAIVDVITEEMLADKAALVVEVMEENYHDAPIVGIALVNEHGRFFMRPETALADSQFLAWLADETKKKSMFDAKRAVVALKWKGIELRGVAFDLLLAAYLLNPAQDAGDIAAVAKMKQYEAVRSDEAVYGKGVKRSLPDEQTLAEHLVRKAAAIWALEQPFMDDLRNNEQDQLLTKLEQPLAAILAEMEFTGVNVDTKRLEQMGSELAEQLRAIEQRIYELAGQEFNINSPKQLGVILFEKLQLPVLKKTKTGYSTSADVLEKLAPHHEIVENILHYRQLGKLQSTYIEGLLKVVRPDTGKVHTMFNQALTQTGRLSSAEPNLQNIPIRLEEGRKIRQAFVPSEPDWLIFAADYSQIELRVLAHIADDDNLIEAFQRDLDIHTKTAMDIFHVSEEEVTANMRRQAKAVNFGIVYGISDYGLAQNLNITRKEAAEFIERYFASFPGVKQYMENIVQEAKQKGYVTTLLHRRRYLPDITSRNFNVRSFAERTAMNTPIQGSAADIIKKAMIDLAARLKEEQLQARLLLQVHDELILEAPKEEIERLCELVPEVMEQAVTLRVPLKVDYHYGPTWYDAK

(19) United States
(12) Patent Application Publication
(10) Pub. No.: US 2015/0315597 A1
(43) Pub. Date: Nov. 5, 2015

(54) Synthetic Nucleic Acids for Polymerization Reactions

(71) Applicant: New England Biolabs, Inc., Ipswich, MA (US)

(72) Inventors: Jennifer Ong, Salem, MA (US); Donald Johnson, Brookline, MA (US); Thomas C. Evans, JR., Topsfield, MA (US); Lucia Greenough, Ipswich, MA (US)

(73) Assignee: NEW ENGLAND BIOLABS, INC., Ipswich, MA (US)

(21) Appl. No.: 14/797,733

(22) Filed: Jul. 13, 2015

Related U.S. Application Data
(63) Continuation-in-part of application No. 13/835,399, filed on Mar. 15, 2013, now Pat. No. 9,109,226.
Application No. 13/835,399 is a continuation-in-part of application No. 13/823,811, filed on Mar. 15, 2013.
(60) Provisional application No. 61/605,484, filed on Mar. 1, 2012.
(60) Provisional application No. 61/530,273, filed on Sep. 1, 2011.
(60) Provisional application No. 61/623,110, filed on Apr. 12, 2012.

(51) Int. Cl. C12N 15/113; C12N 9/12

(52) U.S. Cl. CPC C12N 15/1137; C12N 9/1241; C12N 2320/51; C12N 2310/315; C12N 2310/531

(57) ABSTRACT

Compositions and methods are provided for inhibiting a DNA binding enzyme from reacting with non-target DNA at a temperature below the reaction temperature. The inhibitor is a synthetic nucleic acid which is single stranded but may fold to form at least one double stranded region designed to melt at a temperature which is lower than the reaction temperature, and at least one single stranded region where the single stranded region at the 5′ end contains at least one unnatural and/or modified nucleotide and optionally a sequence at the 3′ end contains one or more derivative nucleotide or linkages.

### CROSS REFERENCE

This application is a continuation-in-part of U.S. patent application Ser. No. 13/835,399, filed Mar. 15, 2013 which is a continuation-in-part of U.S. patent application Ser. No. 13/823,811, filed Mar. 15, 2013. The present application also claims right of priority to U.S. provisional patent application Ser. No. 61/623,110 filed Apr. 12, 2012.

### BACKGROUND

Non-specific primer extension prior to reaction initiation in thermocycling DNA amplification reactions such as polymerase chain reaction (PCR), or isothermal DNA amplification reactions such as loop-mediated isothermal amplification (LAMP) may inhibit specific product formation, and lead to non-specific amplification and reaction irreproducibility. It is, therefore, desirable to block the activity of the polymerase, and hence primer extension, prior to reaction initiation. This has been achieved using antibodies (Kellogg, et al., Biotechniques, 16(6):1134-7 (1994)), affybodies (Affibody AB, Stockholm, Sweden), aptamers (Dang, et al., Journal of Molecular Biology, 264(2):268-78 (1996)), and chemical modification of the polymerase (U.S. Pat. No. 6,183,998). Although each of these techniques can be effective, they each have unique limitations. For example, preparation of antibodies requires use of animal systems, affybodies and aptamers require screening libraries of molecular variants, and chemical modifications require extra heat incubation steps to reverse the inactivating modification. It would be desirable to have a generalizable approach to rapidly and effectively create hot-start inhibitors targeted towards DNA polymerases.

### SUMMARY

In general, a method of reversibly inhibiting a DNA enzyme catalyzed reaction is provided that includes: (a) adding to a mixture comprising a target DNA, a preparation of a DNA enzyme that is active at a temperature of at least 50° C. and an oligonucleotide having a double stranded region and a 5′ overhang wherein a modified or unnatural synthetic nucleotide is located in (i) the double stranded region having a melting temperature (Tm) of less than 50° C. or a Tm at which the DNA enzyme of in the mixture is active; or (ii) the 5′ overhang; and (b) maintaining the mixture at a temperature below the Tm of the double stranded portion of the oligonucleotide to inhibit the DNA enzyme and optionally reversing inhibition by increasing the temperature above the Tm of the double stranded portion to at least 50° C. where the DNA enzyme retains activity at the increased temperature.

In one aspect, the DNA enzyme is for example, a DNase, TET protein, endonuclease, exonuclease, glycosylase, or polymerase. The DNA enzyme includes an enzyme fused to a DNA binding domain.

In one aspect, the DNA enzyme is a polymerase for example, an archael polymerase or a bacterial polymerase such as a variant of a wild type thermostable polymerase. In another aspect, the polymerase has an amino acid sequence that is at least 93% identical to SEQ ID NO:25 and may additionally include at least amino acid substitution at an position corresponding to 278, 307, and/or 402 in SEQ ID NO:25.

In one aspect, the oligonucleotide and the DNA enzyme are present in the solution at a molar ratio of between 0.5:1 to 10:1 and may further comprise dNTPs and primers. In one aspect, the double-stranded region of the oligonucleotide is 4-40 nucleotides in length. In another aspect, the double-stranded region is 6-60 nucleotides in length. In another aspect, the non-standard and/or modified nucleotide is positioned at the fourth position in the 5′ single-strand overhang, numbered from the 3′ end of single-stranded portion of 5′ overhang. In another aspect, the overhang comprises at least 2 non-standard and/or modified or 4 modified nucleotides. In another aspect, the oligonucleotide includes a non-standard and/or modified nucleotide that makes the 3′ end not extendible. Examples of non-standard and/or modified nucleotides include a dideoxynucleotide, inverted base or amino-modified nucleotide at the 3′ end. In one example, the oligonucleotide comprises a linkage or modified nucleotide that makes the 3′ end resistant to nuclease activity. In another example, the oligonucleotide comprises a phosphorothioate linkage at or near the 3′ end.

In general, in one aspect, an aqueous solution is provided that includes (a) a DNA enzyme that is active at a temperature of at least 65° C.; or less than 65° C. or 55° C., or less than 45° C. or 37° C. and (b) an oligonucleotide having a double stranded region and a 5′ overhang wherein a modified or non-standard nucleotide is located in the double stranded region having a Tm of denaturation in the range of 37° C. to 50° C. or below a temperature at which the DNA enzyme is active; or in the 5′ overhang.

In another aspect, the oligonucleotide is capable of inhibiting the DNA enzyme when the aqueous solution is at a temperature below the Tm of the active DNA enzyme.

In general in one aspect, an aqueous solution, includes (a) a thermostable polymerase that is active at a temperature of at least 65° C.; or less than 65° C. or 55° C.; or less than 45° C. or 37° C. and (b) an oligonucleotide that includes (i) a double-stranded region having a Tm selected from a temperature that is less than 65° C., (ii) a 5′ overhang comprising at least one uracil or inosine, and (iii) a modified nucleotide or linkage that makes the 3′ end non-extendible or resistant to nuclease activity; wherein the oligonucleotide is capable of inhibiting the thermostable polymerase when the aqueous solution is at a temperature of below 37° C. but not at a temperature of 65° C. or greater.

In general, in one aspect, a variant of a wild type polymerase comprising at least 93% sequence identity to SEQ ID NO:25 and further comprising at least one mutation at an amino acid position corresponding to 278, 307, and/or 402 in SEQ ID NO:25. The polymerase variant may be fused to a DNA binding domain, for example Sso7d. The polymerase variant may have an amino acid mutation at one or more of the positions corresponding to 278, 307, and/or 402 is not a histidine and optionally fused to a DNA binding protein. For example, the mutation may include one or more mutations selected from a group of mutations corresponding to H278Q, H307R, H402Q, and optionally fused to a DNA binding protein.

Embodiments may include one or more of the following features:

the at least one double-strand region has a Tm of at least 10° C. less than a Tm for a target DNA in an amplification reaction, for example, below 90° C., 89° C., 88° C., 87° C., 86° C., 85° C., 75° C., 65° C., 55° C., 45° C. or 35° C.; a uracil or inosine is positioned at the fourth position in the 5′ single-strand extension numbered from the 3′end; the synthetic nucleic acid is capable of forming a plurality of single-strand regions; a second single-strand region is a spacer; a third single-strand region forms a single-stranded loop at an internal location in the synthetic nucleic acid; the buffer may contain at least one of a polymerase, dNTPs, or primers; the spacer comprises hexa-ethylene glycol, a 3 carbon molecule or a 1′,2′-dideoxyribose; the synthetic nucleic acid contains a derivative nucleotide and/or nucleotide linkage in a nucleic acid sequence at the 3′ end where the derivative nucleotide may be selected from one or more inverted nucleotides, di-deoxynucleotides or amino-modified nucleotides; for example, the nucleotide linkage may be a phosphorothioate linkage.

In an embodiment, the preparation may additionally include one or more polymerases for example, one or more thermostable polymerases, for example at least one archaeal polymerase; a bacterial polymerase, and/or a variant of a wild type archaeal or bacterial polymerase. The synthetic nucleic acid and the polymerase may be present in a molar ratio of between 0.5:1 to 10:1.

In general in one aspect, a variant of a wild type polymerase includes at least 93% sequence identity to SEQ ID NO:25 and further includes at least one mutation at an amino acid position corresponding to 278, 307, and/or 402 in SEQ ID NO:25. In another aspects, mutations at 278, 307 and/or 402 may be inserted into any of the Bst polymerase variants described in U.S. application Ser. No. 13/823,811.

Embodiments of the methods and/or compositions including DNA enzyme variants may include one or more of the following features in a oligonucleotide of the preparation: fusion of variant polymerase to a DNA binding domain such as Sso7d; and/or the variant polymerase optionally having an amino acid at one or more of the positions corresponding to 278, 307, and/or 402 that is not a histidine; for example where one or more mutations may be selected from a group of mutations corresponding to H278Q, H307R, H402Q.

In general in one aspect, a method is provided for inhibiting a polymerase extension reaction; that includes adding a preparation described above to a mixture containing a polymerase, a target DNA and dNTPs; and maintaining for a period of time prior to extension or amplification of the target DNA, the mixture at a temperature below the Tm of the double-stranded portion of the synthetic nucleic acid.

Embodiments may include one or more of the following features:

at least one double-strand region has a Tm of at least 10° C. less than a Tm for a target DNA in an amplification reaction, for example, below 90° C., 89° C., 88° C., 87° C., 86° C., 85° C., 75° C., 65° C., 55° C., 45° C. or 35° C.; a uracil or inosine is positioned at the fourth position in the 5′ single-strand extension numbered from the 3′end; the synthetic nucleic acid may include additional single-stranded nucleic acid regions such as a second single-strand region is a spacer; where for example, the spacer may include a hexa-ethylene glycol, a 3 carbon molecule or a 1′,2′-dideoxyribose; and/or a third single-strand region forms a single-stranded loop at an internal location in the synthetic nucleic acid.

In an embodiment, the synthetic nucleic acid/oligonucleotide contains at least one derivative nucleotide and/or nucleotide linkage at the 3′ end where the at least one derivative nucleotide may be selected from one or more inverted nucleotides, di-deoxynucleotides or amino-modified nucleotides; and for example, the at least one nucleotide linkage may be a phosphorothioate linkage.

In an embodiment, the one or more polymerases may include one or more thermostable polymerases, for example at least one archaeal polymerase; a bacterial polymerase, and/or a variant of a wild type archaeal or bacterial polymerase; and the synthetic nucleic acid and the polymerase may be present in a molar ratio of between 0.5:1 to 10:1.

In one embodiment, an additional step may be included of reversing the inhibition of the polymerase extension reaction by raising the reaction temperature above a Tm for the synthetic nucleic acid.

### BRIEF DESCRIPTION OF THE DRAWINGS

FIG. 1 shows a synthetic nucleic acid in the form of a hairpin oligonucleotide containing a 5′ overhang, a 3′ blocked end to prevent DNA polymerase extension and exonuclease cleavage and at least one non-standard base. (1) is the optional spacer; (2) is the double-stranded region or “stem”; (3) is the 5′ single-strand; and (4) is the blocked 3′ end: N=rNMP, dNMP or non-standard base; X=base that is recognized by the DNA polymerase uracil binding pocket; *=3′ end modifications: phosphorothioate bonds and/or inverted base and/or dideoxynucleoside.

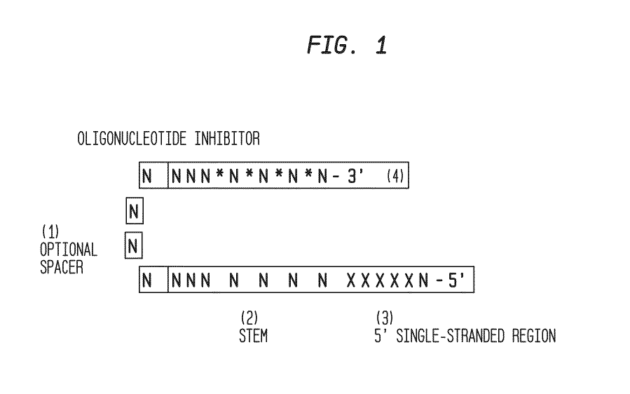

FIG. 2 shows a gel of the PCR products obtained with an Archaeal polymerase in the presence or absence of hairpin oligonucleotide inhibitors. In the absence of the hairpin oligonucleotide, the polymerase fails to amplify the expected 2 kb product. In the presence of the oligonucleotides the 2 kb product is amplified.

Lane 1 contains 2-log DNA ladder (New England Biolabs, Ipswich, Mass.), a MW marker for detection of 2 Kb amplicon.

Lane 2 contains 5 nM Archaeal Family B DNA polymerase without the synthetic nucleic acid present.

Lane 3 contains 5 nM Archaeal Family B DNA polymerase and 5 nM the synthetic nucleic acid, TM39U1G-Is.

Lane 4 contains 5 nM Archaeal Family B DNA polymerase and 5 nM the synthetic nucleic acid, TM39U1G-I*.

Lane 5 contains 5 nM Archaeal Family B DNA polymerase and 5 nM the synthetic nucleic acid, TM39U.

Lane 6 contains 5 nM Archaeal Family B DNA polymerase and 5 nM the synthetic nucleic acid, TM39Loop10T.

Lane 7 contains 5 nM Archaeal Family B DNA polymerase and 5 nM the synthetic nucleic acid, TM39U3-Is.

### DETAILED DESCRIPTION OF THE EMBODIMENTS

Synthetic nucleic acids/oligonucleotides are described that reversibly inhibit DNA binding enzyme reactions. These synthetic nucleic acid preferably contain at least one non-standard and/or modified base in a 5′ single-strand overhang adjacent to a double-strand region. If the double-strand region is denatured into a single-strand or strands, the synthetic nucleic acid no longer blocks the DNA binding enzyme from reacting with substrate DNA. Preferably, inhibition of the DNA binding enzyme activity occurs at a first temperature that is at least 10° C. lower than a second temperature suitable for enzyme reactions. Where the DNA enzyme is a polymerase, a polymerase extension reaction refers to the extension of a first single-strand nucleic acid by a polymerase where the extension is complementary to a second nucleic acid in association with the first strand.

The term “Tm” refers to a computer predicted 50% denaturation temperature for a double stranded nucleic acid based on nucleotide sequence composition. Examples of computer based predictors include UnaFold (Integrated DNA technologies Coralville, Iowa) and Mfold Webserver, (The RNA Institute, College of Arts and Sciences, University of Albany, State University of NY).

In an embodiment, a synthetic nucleic acid is engineered so that the double-strand region has a Tm at a desired temperature for degeneration at about 15° C. or 14° C. or 13° C. or 12° C. or 11° C. or 10° C. or 9° C. or 8° C. below DNA binding enzyme reaction conditions. Where the DNA binding enzyme is a polymerase, the conditions of the reaction, namely polymerase extension reaction conditions include conditions for isothermal amplification occurring at for example 65° C. or a thermocycling amplification such as PCR which occurs at higher temperatures such as about 95° C.

The DNA binding protein may be a product of fusion to a DNA binding domain. Additional the DNA binding protein may be fused to a protein moiety other than a DNA binding domain, wherein the second protein moiety may be associated with solubility, e.g. maltose binding protein (MBP) or purification such as MBP or chitin binding domain (CBD).

Preferably, the double-strand region in the synthetic nucleic acid is designed to remain intact at a specific temperature in the range of −80° C. to 37° C. but become denatured at a specific temperature which exceeds the temperature at which it remains intact where the denaturation temperature is in a range of 37° C. to 100° C. The Tm of the synthetic nucleic acid can be modulated by one or more factors that include: changing the sequence or length of the double-strand region, changing the length of an internal single-strand region, adding mismatched, non-standard and/or modified bases to the double-strand region, selecting a nucleotide composition having weaker base pairing properties such as an adenine, thymine or uracil rich sequence, and having a sequence containing inosine, or abasic sites such as 1′,2′ dideoxyribose in a polymerization reaction buffer with a selected salt type (for example magnesium) and concentration. An example of a polymerization reaction buffer is Thermopol® Buffer (New England Biolabs, Ipswich, Mass.).

In an embodiment of the invention, the design of a synthetic nucleic acid reversible inhibitor of polymerase extension reactions includes the following features: the synthetic nucleic acid can be DNA, DNA/RNA, RNA, or RNA/RNA; it can be formed from two single-strands or from a single nucleic acid (oligonucleotide) but should be capable of forming a molecule or a plurality of molecules comprising at least one double-strand region and a 5′ single-strand overhang. The synthetic nucleic acid inhibitor may optionally contain a plurality of single-strand regions and a plurality of double-strand regions. If the synthetic nucleic acid inhibitor is an oligonucleotide, it should be capable of folding in such a way as to contain at least one double-strand region at a temperature lower than the reaction temperature as described above. The oligonucleotide may have a length in the range of 8-200 nucleotides. Any double-strand region in the inhibitor preferably has a length of 4-35 nucleotides.

The term “Modified nucleotide” refers to any of ATGC with an additional chemical group attached to the nucleotide such as a methyl, hydroxymethyl, formyl or carboxy group. It includes a non-standard and/or modified nucleotide such as for example might be observed in damaged DNA such as 8-oxo-G or Uracil or to an unnatural synthetic nucleotide such as benzylguanine.

The 5′ single-strand region such as an overhang should be at least 4 nucleotides and preferably less than 100 nucleotides in length, for example 4-40 nucleotides, for example 6-10 nucleotides, and should contain one or more non-standard and/or modified nucleotides such as U or I positioned between the second and tenth position of the overhang counted from the double-strand region, for example in the fourth position where the one or more non-standard and/or modified nucleotides may be 1 to 5 uracils or 1 to 5 inosines. For example, the sequences shown in Table 1 were all found to be effective as reversible binding oligonucleotides.

In addition, a synthetic nucleic acid may optionally have a 3′ end that is resistant to exonuclease activity and/or non-extendable by a polymerase. The 3′ end of the oligonucleotide can be blocked from extension by modification, such as dideoxynucleotides, spacer molecules, inverted bases or amino-modified nucleotides. The 3′ end can be made resistant to exonuclease degradation by the addition of phosphorothioate linkages between one or more bases at or near the 3′ end or the use of inverted bases at the 3′ end. The oligonucleotide can be made non-amplifiable by adding non-replicable bases in the internal sequence, such as carbon spacers, 1′,2′-Dideoxyribose, abasic site, or thymine dimers.

Table 1 provides examples of synthetic nucleic acid molecules capable of forming hairpins and that were found to be effective in the assays described herein. The exemplified synthetic nucleic acid molecules have spacers of Tₙ or Xₙ where T₍₄₋₉₎ or X₍₁₋₄₎, a 5′ end containing a modified base, U₍₁₋₅₎, or I₍₁₋₃₎ and has a U or an I at position 4 counted from the double-stranded region. The 5′ end varies as shown.

TABLE 1
Oligonucleotides tested and effective in Hot Start PCR

| Oligo | |
|---|---|
| Length | Sequence containing uracil (U) or Inosine (I) |
| 28 | TUUUUUCTATCCTTAAGGA*T*A*G (SEQ ID NO: 3) |
| 24 | TUUUUUAGCTAGGTTTTCCTA*G*C*T (SEQ ID NO: 4) |
| 24 | TUUUUUGCAGCGATTTTTCGC*T*G*C (SEQ ID NO: 5) |
| 30 | TUUUUUGAGACTCGRCTTTTGACGAGT*C*T*C (SEQ ID NO: 6) |
| 34 | TUUUUUCTATCCTTAACGTTTTCGTTAAGGA*T*A*G (SEQ ID NO: 7) |
| 30 | TUUUUUACACTTCCGGTTTTCCGGAAG*T*G*T (SEQ ID NO: 8) |
| 31 | TUUUUUCTATCCTTAACGXCGTTAAGGA*T*A*G (SEQ ID NO: 9) |
| 34 | TUUUUUCTATCCTTAACGXXXXCGTTAAGGA*T*A*G (SEQ ID NO: 10) |
| 36 | TUUUUUCTATCCTTAACGTTTTTTCGTTAAGGA*T*A*G (SEQ ID NO: 11) |
| 40 | TUUUUUCTATCCTTAACGTTTTTTTTTTCGTTAAGGA*T*A*G (SEQ ID NO: 12) |
| 34 | TUUUUUCTATCCTTAACITTTTCGTTAAGGA*T*A*G (SEQ ID NO: 13) |
| 34 | TUUUUUCTATCCTTAACITTTTCGTTAAGG*A*T*A*G (SEQ ID NO: 14) |
| 34 | TUUUUUATCTCCTTAACITTTTCGTTAAGGAGAinvdT(SEQ ID NO: 15) |
| 34 | TUUUUUCTITCCTTIICGTTTTCGTTAAGGA*T*A*G (SEQ ID NO: 16) |
| 34 | TAUGGACTATCCTTAACGTTTTCGTTAAGGA*T*A*G (SEQ ID NO: 17) |
| 34 | TUUUGACTATCCTTAACGTTTTCGTTAAGGA*T*A*G (SEQ ID NO: 18) |
| 34 | TTITTTCTATCCTTAACGTTTTCGTTAAGGA*T*A*G (SEQ ID NO: 19) |
| 34 | TTITTTCTATCCTTAACGTTTTCGRRAAGG*A*T*A*G (SEQ ID NO: 20) |
| 34 | TTITTTATCTCCTTAACGTTTTCGRRAAGGAGAinvdT(SEQ ID NO: 21) |
| 34 | TIIITTCTATCCTTAACGTTTTCGTTAAGGA*T*A*G (SEQ ID NO: 22) |
| 34 | TIIITTCTATCCTTAACGTTTTCGTTAAGG*A*T*A*G (SEQ ID NO: 23) |
| 34 | TIIITTATCTCCTTAACGTTTTCGTTAAGGAGAinvdT(SEQ ID NO: 24) |

* = phosphorothoate bonds

In an embodiment of the invention, one or more DNA binding enzymes are added to the synthetic nucleic acid. Examples of DNA binding enzymes include: DNAses, TET, endonucleases, exonucleases, glycosylases and polymerases. The design of the oligonucleotide inhibitor is intended to inhibit the enzyme activity unless the oligonucleotide is denatured by temperature or chemical agent under conditions that do not inactivate the DNA binding enzyme in which case, the inhibition is reversed.

In one embodiment, the DNA enzyme may be a polymerase. Examples of polymerases include thermostable polymerases such as wild type or recombinant Archaeal DNA polymerases or bacterial DNA polymerases or variants (mutants) thereof including fusion proteins where the polymerase or variants thereof may be fused to a DNA binding domain such as Sso7d (for example, U.S. Pat. No. 7,666,645). A variant of a bacterial polymerase is exemplified at least 90%, 91%, 92% 93%, 95%, or 98% amino acid sequence homology or identity with SEQ ID NO:25 prior to fusion to a DNA binding domain if such is present. Regardless of the presence of an additional DNA binding domain, the variant preferably includes one or more mutations at positions corresponding to 52 (not R), 278, 307, 402, and/or 578 (not R) in SEQ ID NO:25, for example, one or more of the following mutations: H278Q, H307R, H402Q. Additional mutations may be optionally introduced into the polymerase by routine methods of random or directed mutagenesis. The examples demonstrate this effect with an oligonucleotide used together with an archael polymerase which is inhibited by the oligonucleotide until the temperature is raised to a level at which the oligonucleotide is denatured and the polymerase is active.

Amplification procedures referred to herein include standard thermocycling or isothermal amplification reactions such as PCR amplification or LAMP (Gill, et al., Nucleos. Nucleot. Nucleic Acids, 27:224-43 (2008); Kim, et al, Bioanalysis, 3:227-39 (2011); Nagamine, et al., Mol. Cel. Probes, 16:223-9 (2002); Notomi, et al., Nucleic Acids Res., 28:E63 (2000); and Nagamine, et al., Clin. Chem., 47:1742-3 (2001)), helicase displacement amplification (HDA), recombinase polymerase amplification (RPA), nicking enzyme amplification reaction (NEAR) and/or strand displacement amplification (SDA). Variant polymerases described herein may be used in amplification or sequencing reactions with or without the use of synthetic nucleic acids described herein.

Amino acid sequence for a wild type Bst polymerase:

(SEQ ID NO: 25)
AEGEKPLEEMEFAIVDVITEEMLADKAALVVEVMEENYHDAPIVGIALVN
EHGRFFMRPETALADSQFLAWLADETKKKSMFDAKRAVVALKWKGIELRG
VAFDLLLAAYLLNPAQDAGDIAAVAKMKQYEAVRSDEAVYGKGVKRSLPD
EQTLAEHLVRKAAAIWALEQPFMDDLRNNEQDQLLTKLEQPLAAILAEME
FTGVNVDTKRLEQMGSELAEQLRAIEQRIYELAGQEFNINSPKQLGVILF
EKLQLPVLKKTKTGYSTSADVLEKLAPHHEIVENILHYRQLGKLQSTYIE
GLLKVVRPDTGKVHTMFNQALTQTGRLSSAEPNLQNIPIRLEEGRKIRQA
FVPSEPDWLIFAADYSQIELRVLAHIADDDNLIEAFQRDLDIHTKTAMDI
FHVSEEEVTANMRRQAKAVNFGIVYGISDYGLAQNLNITRKEAAEFIERY
FASFPGVKQYMENIVQEAKQKGYVTTLLHRRRYLPDITSRNFNVRSFAER
TAMNTPIQGSAADIIKKAMIDLAARLKEEQLQARLLLQVHDELILEAPKE
EIERLCELVPEVMEQAVTLRVPLKVDYHYGPTWYDAK

All references cited herein are incorporated by reference.

### Example

### Assay to Measure Inhibition of Polymerase Activity Prior to PCR Cycling

Inhibition of polymerase activity was measured at a temperature below that used in the PCR assay which followed.

The assay was performed as follows:

Primers were made for PCR to produce a 2 kb Lambda DNA amplicon. Additionally, the 3′ end of the reverse primer contained 8 nucleotides that could anneal to Lambda DNA creating a false priming site producing a non-specific 737 bp amplicon.

The PCR assay was done in the presence of high levels of human genomic DNA and the reaction mixture was incubated with the thermostable polymerase at 25° C. for 15 minutes prior to PCR cycling. These conditions created many opportunities to form non-specific products. The presence of a nucleic acid composition to inhibit polymerase activity prior to amplification was required to yield a 2 kb amplicon, with minimal or no non-specific products. The reaction mix was set up on ice and contained the following reagents: Thermopol Buffer, 0.4 pg/μl Lambda DNA, 2.0 ng/μl Jurkat genomic DNA, 0.2 mM dNTP and 0.2 μM primers.

Forward primer, L30350F:
(SEQ ID No: 1)
5′CCTGCTCTGCCGCTTCACGC3′
Reverse primer, L2kbalt4rv:
(SEQ ID No: 2)
5′GGGCCGTGGCAGTCGCATCCC3′

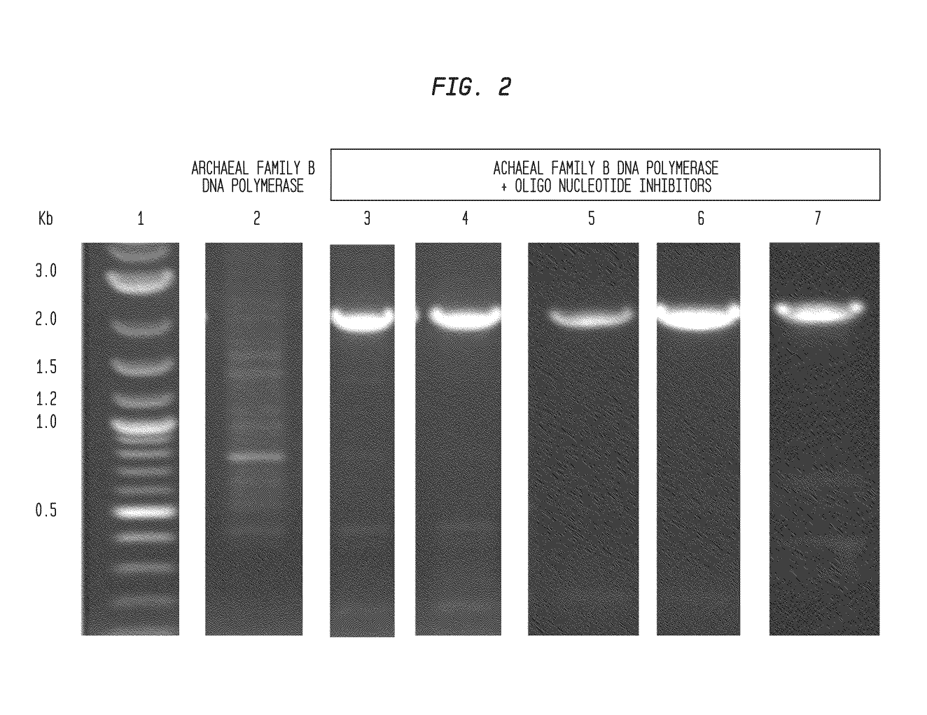

0.25 μl to 0.50 μl of 2.0 units/μl Vent® DNA Polymerase (NEB, Ipswich, Mass.) with or without the nucleic acid composition (see FIG. 2) was added to 25 μl or 50 μl of the reaction mix, and transferred to a PCR machine and cycled at 25° C. for 15-30 minutes, then cycled 35 times at 98° C. for 10 seconds, 45° C. for 20 seconds, 72° C. for 60 seconds, 72° C. for 4 minutes. DNA products generated by PCR cycling were analyzed by agarose gel electrophoresis.

In the absence of a reversibly inhibiting synthetic nucleic acid, the polymerase failed to yield the expected 2 kb Lamda amplicon. Non-specific products including the 737 bp amplicon were observed. In the presence of oligonucleotide inhibitors, a robust yield of the expected 2 kb Lambda amplicon was produced with minimal or no non-specific products.

What is claimed is:

1. A method of reversibly inhibiting a DNA enzyme catalyzed reaction; comprising:
(a) adding to a mixture comprising a target DNA, a preparation of a DNA enzyme optionally fused to a second protein, the DNA enzyme being active at a temperature of at least 50° C. and an oligonucleotide having a double stranded region and a 5′ overhang wherein a non-standard and/or modified nucleotide is located in:
i. the double stranded region having a melting temperature (Tm) of less than 50° C. or a Tm at which the DNA enzyme of (a) is active; or
ii. the 5′ overhang.
(b) maintaining the mixture at a temperature below the Tm of the double stranded portion of the oligonucleotide to inhibit the DNA enzyme and optionally reversing inhibition by increasing the temperature above the Tm of the double stranded portion of the oligonucleotide to at least 50° C. where the DNA enzyme retains activity at the increased temperature.

2. The method of claim 1, wherein the DNA enzyme is a polymerase.

3. The method of claim 2, wherein the polymerase is an archael polymerase.

4. The method of claim 2, wherein the polymerase is a bacterial polymerase.

5. The method of claim 2, wherein the polymerase is a variant of a wild type thermostable polymerase.

6. The method of claim 5, wherein the polymerase has an amino acid sequence that is at least 93% identical to SEQ ID NO:25.

7. The method of claim 6, wherein the polymerase has an amino acid sequence comprises at least amino acid substitution at an position corresponding to 278, 307, and/or 402 in SEQ ID NO:25.

8. The method of claim 1, wherein the oligonucleotide and the DNA enzyme are present in the mixture at a molar ratio of between 0.5:1 to 10:1.

9. The method of claim 7, wherein the preparation further comprises dNTPs and primers.

10. The method of claim 1, wherein the double stranded region of the oligonucleotide is 4-40 nucleotides in length.

11. The method of claim 1, wherein the double stranded region of the oligonucleotide is 6-60 nucleotides in length.

12. The method of claim 1, wherein the non-standard and/or modified nucleotide is positioned at the fourth position in the 5′ single-strand overhang, numbered from the 3′ end of single-stranded portion of 5′ overhang.

13. The method of claim 1, wherein the overhang comprises at least 2 non-standard and/or modified or 4 non-standard and/or modified nucleotides.

14. The method of claim 1, wherein the oligonucleotide comprises a non-standard and/or modified nucleotide that makes the 3′ end not extendible.

15. The method of claim 1, wherein the non-standard and/or modified nucleotide is selected from a dideoxynucleotide, inverted base or amino-modified nucleotide at the 3′ end.

16. The method of claim 1, wherein the oligonucleotide comprises a linkage or the non-standard and/or modified nucleotide that makes the 3′ end resistant to nuclease activity.

17. The method of claim 1, wherein the oligonucleotide comprises a phosphorothioate linkage at or near the 3′ end.

18. A method according to claim 1, wherein the oligonucleotide is capable of folding to form a plurality of single-strand regions.

19. A method according to claim 18, wherein a second single-strand region is a spacer.

20. A method according to claim 18, wherein a third single-strand region forms a loop at an internal location in the synthetic nucleic acid.

21. A method according to claim 19, wherein the spacer comprises hexa-ethylene glycol, a 3 carbon molecule or a 1′,2′-dideoxyribose.

22. A method according to claim 1, wherein the 3′ end of the oligonucleotide contains a derivative nucleotide and/or nucleotide linkage.

23. A method according to claim 22, wherein the derivative nucleotide is selected from one or more inverted nucleotides, di-deoxynucleotides or amino-modified nucleotides.

24. A method according to claim 22, wherein the nucleotide linkage is a phosphorothioate linkage.

25. A variant of a wild type polymerase comprising at least 93% sequence identity to SEQ ID NO:25 and further comprising at least one mutation at an amino acid position corresponding to 278, 307, and/or 402 in SEQ ID NO:25.

26. A variant of a wild type polymerase according to claim 25, fused to a DNA binding domain.

27. A variant of a wild type polymerase according to claim 26, wherein the DNA binding domain is Sso7d.

28. A variant of a wild type polymerase according to claim 25, wherein an amino acid at one or more of the positions corresponding to 278, 307, and/or 402 is not a histidine and optionally fused to a DNA binding protein.

29. A variant of a wild type polymerase according to claim 25, further comprising one or more mutations selected from a group of mutations corresponding to H278Q, H307R, H402Q, and optionally fused to a DNA binding protein.
